(19) United States
(12) Patent Application Publication
(10) Pub. No.: US 2024/0197928 A1
(43) Pub. Date: Jun. 20, 2024

(54) SODIUM-DEPENDENT GLUCOSE TRANSPORTER 2 AS A DIAGNOSTIC AND THERAPEUTIC TARGET FOR PRE-MALIGNANT LESIONS

(71) Applicant: THE REGENTS OF THE UNIVERSITY OF CALIFORNIA, OAKLAND, CA (US)

(72) Inventors: Claudio SCAFOGLIO, LOS ANGELES, CA (US); Steven M. DUBINETT, LOS ANGELES, CA (US)

(73) Assignee: THE REGENTS OF THE UNIVERSITY OF CALIFORNIA, OAKLAND, CA (US)

(21) Appl. No.: 17/755,429

(22) PCT Filed: Oct. 28, 2020
(86) PCT No.: PCT/US20/57732

§ 371 Date: Apr. 28, 2022

Related U.S. Application Data
(60) Provisional application No. 62/927,036, filed on Oct. 28, 2019.

(51) Int. Cl. A61K 51/04; A61K 31/7034; A61K 31/7042; A61K 31/7048

(52) U.S. Cl. CPC A61K 51/0491; A61K 31/7034; A61K 31/7042; A61K 31/7048

(57) ABSTRACT

Methods, kits, and compositions are described for the detection and treatment of premalignant lesions. This early detection allows patients to avoid the risks associated with repetitive imaging often employed to seek differentiation between developing cancer and inflammation. The method comprises (a) administering to the subject a radiographic tracer for a sodium/glucose cotransporter (SGLT): (b) performing a radiographic detection scan of the subject; and (c) detecting signal emitted by the tracer taken up in the scanned subject, whereby detected signal in the subject is indicative of a pre-malignant lesion. In some embodiments, the method further comprises administering an inhibitor of sodium-glucose transporter 2 (SGLT2), such as gliflozin, to a subject in whom a pre-malignant lesion has been detected. In some embodiments, the pre-malignant lesion is in tissue that expresses SGLT2, such as a lung, prostate, bladder, breast, or pancreatic lesion.

This application claims benefit of U.S. provisional patent application No. 62/927,036, filed Oct. 28, 2019, the entire contents of which are incorporated by reference into this application.

### ACKNOWLEDGEMENT OF GOVERNMENT SUPPORT

This invention was made with government support under Grant Number TR001881, awarded by the National Institutes of Health. The government has certain rights in the invention.

### REFERENCE TO A SEQUENCE LISTING SUBMITTED VIA EFS-WEB

The content of the ASCII text file of the sequence listing named “UCLA273_seq”, which is 1 kb in size was created on Oct. 28, 2020, and electronically submitted via EFS-Web herewith the application, is incorporated herein by reference in its entirety.

### BACKGROUND

Non-small cell lung cancer (NSCLC) is the leading cause of cancer-related mortality worldwide. Despite the success of targeted and immune-based therapies in NSCLC, early diagnosis and surgical resection of early-stage disease remain the best opportunity for a cure: the 5-year survival of NSCLC patients is 55.6% for localized disease but only 4.5% for metastatic disease. However, according to statistics of the NIH Surveillance, Epidemiology, and End Result Program, only 16% of newly diagnosed lung cancers are localized, whereas the majority has already spread to regional lymph nodes or to distant metastatic sites at the time of diagnosis. Recently, intensive research efforts have been directed to the elucidation of the molecular mechanisms of pulmonary premalignancy development and progression to find signatures of premalignancy that can be targeted for early diagnosis and cancer chemoprevention and/or interception.

Lung adenocarcinoma (LADC) and squamous cell carcinoma (SqCC) are the most frequent histologic subtypes of NSCLC. The sequence of pre-malignant lesions that leads to the development of SqCC has been extensively described and studied because SqCC develops from the respiratory epithelium of large airways that are accessible by bronchoscopy. Conversely, LADC develops mostly within terminal bronchioles and alveoli, which are not readily accessible by bronchoscopy. Therefore, LADC premalignancy has been more elusive; the only pre-malignant lesion known to be a precursor of LADC is atypical adenomatous hyperplasia (AAH), consisting of a localized growth of pre-malignant, cuboidal cells lining the alveolar walls, defined as a lepidic pattern. AAH can progress to adenocarcinoma in situ (AIS) and minimally invasive adenocarcinoma (MIA), both of which are precursors of invasive adenocarcinoma and are also characterized by lepidic growth. AAH, AIS, and MIA can be detected in vivo by high-resolution computed tomography (CT), typically presenting as pure or predominantly ground-glass nodules (GGNs). However, CT is not specific, and GGNs can also correspond to benign lesions, such as alveolar inflammation and hemorrhage. Thus, biomarkers to aid the diagnostic definition of GGNs and to identify AAH, AIS, and MIA non-invasively are urgently needed.

### SUMMARY

The methods described herein provide for the detection and treatment of pre-malignant lesions. Using the methods described herein, it is now possible to detect pre-malignant lesions that cannot otherwise be detected using conventional methods, such as PET scans or CT scans. This early detection allows patients to avoid the risks associated with repetitive imaging often employed to seek differentiation between developing cancer and inflammation. In addition, these pre-malignant lesions can be targeted therapeutically using the treatment methods described herein, greatly improving outcomes for patients.

In some embodiments, described herein is a method of detecting pre-malignant lesions in a subject. In some embodiments, the method comprises (a) administering to the subject a radiographic tracer for a sodium/glucose cotransporter (SGLT); (b) performing a radiographic detection scan of the subject; and (c) detecting signal emitted by the tracer taken up in the scanned subject. Detected signal in the subject is indicative of a pre-malignant lesion. In some embodiments, the tracer comprises a C 1-O-methyl or ethyl pyranoside having an equatorial hydroxyl group at carbon-2, radiolabeled with ¹⁸F, ¹²³I, or ¹²⁴I or a free hexose having an equatorial hydroxyl group at carbon-2, radiolabeled with ¹⁸F, ¹²³I, or ¹²⁴I. In some embodiments, the tracer comprises methyl-4-deoxy-4-[¹⁸F]fluoro-D-glucopyranoside (Me4FDG), 1-[¹⁸F]fluoro-1-deoxy-D-glucose (“1-FDG”), or 4-[18F]fluoro-dapagliflozin. These and additional tracers are described in U.S. Pat. No. 8,845,999, and in Ghezzi, et al., J Am Soc Nephrol. 2017 March; 28 (3):802-810.

In some embodiments, the radiographic detection scan is a positron emission tomography (PET) scan. In some embodiments, the detecting comprises calculating a contrast to noise ratio (CNR) of a PET signal. In some embodiments, the method further comprises administering an inhibitor of sodium-glucose transporter 2 (SGLT2) to a subject in whom a pre-malignant lesion has been detected. In some embodiments, the subject is a human.

In some embodiments, the pre-malignant lesion is a lesion in tissue that expresses SGLT2. In some embodiments, the lesion is a lung lesion. In some embodiments, the lung lesion comprises atypical adenomatous hyperplasia (AAH), adenocarcinoma in situ, invasive adenocarcinoma, and/or minimally invasive adenocarcinoma. In some embodiments, the lesion is a prostate, bladder, breast, or pancreatic lesion.

Further described herein is a method of inhibiting the development or progression of a pre-malignant lesion in a subject, comprising administering to the subject an inhibitor of SGLT2. In some embodiments, the subject has a pre-malignant lesion. In some embodiments, the subject has a pre-malignant lesion detected by a method described herein. In some embodiments, the subject is suspected of having, and/or is at high risk of having, a pre-malignant lesion. In some embodiments, the inhibitor is administered in a therapeutically effective amount. In some embodiments, the inhibitor is a gliflozin. In some embodiments, the gliflozin is dapagliflozin, canagliflozin, empagliflozin, or ertugliflozin. In some embodiments, the method of detection described herein is performed after treatment to confirm efficacy and/or to monitor treatment and recovery.

### BRIEF DESCRIPTION OF THE DRAWINGS

FIG. 1 Images demonstrating that Me4FDG can detect lung lesions that are negative for FDG uptake. A patient with multiple lung lesions received high-resolution CT scan (upper panel), FDG PET/CT (middle panel) and Me4FDG PET/CT (lower panel). The circle indicates a ground glass opacity, a kind of lesion typically corresponding to lesions of the lung adenocarcinoma spectrum, which is negative for FDG but positive for Me4FDG uptake.

FIG. 2 Images from Me4FDG PET/CT and immunohistochemistry, comparing two lesions in human patients, one positive and one negative with Me4FDG (circles in A). The corresponding pathological specimens were stained with immunohistochemistry to measure the expression of SGLT2 in the lesion. The dark signal indicates the presence of the SGLT2 transporter in the lesion. Both lesions are adenocarcinomas, but while the Me4FDG positive one is positive for SGLT2, the negative one does not have the SGLT2 protein (immunostaining in B). This observation confirms that Me4FDG recognizes the lesions that are malignant and rely on SGLT2 for glucose uptake.

### DETAILED DESCRIPTION

The invention described herein is based on the discovery that the sodium-dependent glucose transporter SGLT2 is specifically expressed in lung pre-malignancy and early-stage lung cancer, and is a mechanism of metabolic supply required for the early stages of lung cancer development. As demonstrated herein, SGLT2 can be used for early detection of lung nodules in murine models with the positron emission tomography tracer methyl-4-[18F] fluorodeoxyglucose (Me4FDG), a glucose analog that detects specifically transporters of the SGLT family.

Importantly, described herein is the discovery that the tracer Me4FDG can image lesions that are negative with the traditional tracer FDG. This type of lesion has the typical appearance of ground-glass opacity, which can correspond to lung adenocarcinoma pre-malignancy. These observations show that lesions that are pre-malignant, yet negative with the traditional tracer FDG, can now be detected. This also shows that the data from the mouse model (see Example 1 below) is relevant to human patients.

In addition, pharmacological blockade of SGLT2 activity with specific inhibitors can be used as a chemopreventive strategy to prevent the development of advanced cancer in patients with lung nodules corresponding to pre-malignant lesions. Described herein is the use of Me4FDG for early diagnosis of premalignant lesions, coupled with the use of SGLT2 inhibitors as a cancer interception and chemoprevention strategy to prevent the progression of early lesions to invasive cancer. This early diagnosis is especially valuable in the context of lesions or nodules that appear in multiple sites, which would otherwise require clinically impossible multiple biopsies. The method can also be used to monitor disease progression and/or to confirm successful treatment.

Demonstrated herein is further support for the value of SGLT2-based detection and treatment of lesions, via comparison of two lesions in human patients, one positive and one negative with Me4FDG. Both lesions are adenocarcinomas. While the positive lesion is positive for SGLT2, the negative one does not have the SGLT2 protein. This observation confirms that Me4FDG recognizes the lesions that are malignant and rely on SGLT2 for glucose uptake.

### Definitions

All scientific and technical terms used in this application have meanings commonly used in the art unless otherwise specified. As used in this application, the following words or phrases have the meanings specified.

As used herein, a “control” or “reference” sample means a sample that is representative of normal measures of the respective marker, such as would be obtained from normal, healthy control subjects, or a baseline amount of marker to be used for comparison. Typically, a baseline will be a measurement taken from the same subject or patient. The sample can be an actual sample used for testing, or a reference level or range, based on known normal measurements of the corresponding marker.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference sample is a significant difference. In other examples, an increase or decrease of 20%, 30%, 40%, or 50% relative to the reference sample is considered a significant difference. In yet another example, an increase of two-fold relative to a reference sample is considered significant.

The term “effective amount” or “therapeutically effective amount” or “prophylactically effective amount”, or “diagnostically effective amount”, refer to an amount of an active agent described herein that is effective to provide the desired/intended result and/or biological activity. Thus, for example, in various embodiments, an effective amount of a therapeutic agent is an amount that is effective to slow the progression of, and/or to hinder, and/or to reverse tumorigenesis and/or progression to malignancy and/or cancer.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, PA, 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

As used herein, to “prevent” or “protect against” a condition or disease means to hinder, reduce or delay the onset or progression of the condition or disease.

### Methods

The methods described herein provide for the detection and treatment of pre-malignant lesions. Using the methods described herein, it is now possible to detect pre-malignant lesions that cannot otherwise be detected using conventional methods, such as PET scans or CT scans. This early detection allows patients to avoid the risks associated with repetitive imaging often employed to seek differentiation between developing cancer and inflammation. In addition, these pre-malignant lesions can be targeted therapeutically using the treatment methods described herein, greatly improving outcomes for patients.

In some embodiments, described herein is a method of detecting pre-malignant lesions in a subject. In some embodiments, the method comprises (a) administering to the subject a radiographic tracer for a sodium/glucose cotransporter (SGLT); (b) performing a radiographic detection scan of the subject; and (c) detecting signal emitted by the tracer taken up in the scanned subject. Detected signal in the subject is indicative of a pre-malignant lesion. In some embodiments, the tracer comprises a C 1-O-methyl or ethyl pyranoside having an equatorial hydroxyl group at carbon-2, radiolabeled with ¹⁸F, ¹²³I, or ¹²⁴I or a free hexose having an equatorial hydroxyl group at carbon-2, radiolabeled with ¹⁸F, ¹²³I, or ¹²⁴I. In some embodiments, the tracer comprises methyl-4-deoxy-4-[¹⁸F]fluoro-D-glucopyranoside (Me4FDG), 1-[¹⁸F]fluoro-1-deoxy-D-glucose (“1-FDG”), or 4-[18F]fluoro-dapagliflozin. These and additional tracers are described in U.S. Pat. No. 8,845,999, and in Ghezzi, et al., J Am Soc Nephrol. 2017 March; 28 (3):802-810.

In some embodiments, the radiographic detection scan is a positron emission tomography (PET) scan. In some embodiments, the detecting comprises calculating a contrast to noise ratio (CNR) of a PET signal. In some embodiments, the method further comprises administering an inhibitor of sodium-glucose transporter 2 (SGLT2) to a subject in whom a pre-malignant lesion has been detected. In some embodiments, the subject is a human.

In some embodiments, the pre-malignant lesion is a lesion in tissue that expresses SGLT2. In some embodiments, the lesion is a lung lesion. In some embodiments, the lung lesion comprises atypical adenomatous hyperplasia (AAH), adenocarcinoma in situ, invasive adenocarcinoma, and/or minimally invasive adenocarcinoma. In some embodiments, the lesion is a prostate, bladder, breast, or pancreatic lesion.

Further described herein is a method of inhibiting the development or progression of a pre-malignant lesion in a subject, comprising administering to the subject an inhibitor of SGLT2. In some embodiments, the subject has a pre-malignant lesion. In some embodiments, the subject has a pre-malignant lesion detected by a method described herein. In some embodiments, the subject is suspected of having, and/or is at high risk of having, a pre-malignant lesion. In some embodiments, the inhibitor is administered in a therapeutically effective amount. In some embodiments, the inhibitor is a gliflozin. In some embodiments, the gliflozin is dapagliflozin, canagliflozin, empagliflozin, or ertugliflozin. In some embodiments, the method of detection described herein is performed after treatment to confirm efficacy and/or to monitor treatment and recovery.

Treatment of lesions can be administered in a single dose or as a series of doses administered over time. Dosage and treatment regimens can be determined by the treating physician, taking into account disease severity, patient condition, and other factors.

### Kits & Compositions

The invention provides kits and/or compositions comprising one or more reagents and/or therapeutic agents suitable for use in the methods described herein, and optionally, one or more suitable containers containing reagents and/or agents of the invention. Such kits can comprise a carrier, package or container that is compartmentalized to receive one or more containers such as vials, tubes, and the like, each of the container(s) comprising one of the separate elements to be used in the method. The reagents and/or agents of the kit may be provided in any suitable form, including frozen, lyophilized, or in a pharmaceutically acceptable buffer such as TBS or PBS.

Reagents include a radiographic tracer for a sodium/glucose cotransporter (SGLT). In some embodiments, the tracer comprises a C 1-O-methyl or ethyl pyranoside having an equatorial hydroxyl group at carbon-2, radiolabeled with ¹⁸F, ¹²³I, or ¹²⁴ I or a free hexose having an equatorial hydroxyl group at carbon-2, radiolabeled with ¹⁸F, ¹²³I, or ¹²⁴I. In some embodiments, the tracer comprises methyl-4-deoxy-4-[¹⁸F]fluoro-D-glucopyranoside (Me4FDG), 1-[¹⁸F]fluoro-1-deoxy-D-glucose (“1-FDG”), or 4-[18F]fluoro-dapagliflozin. These and additional tracers are described in U.S. Pat. No. 8,845,999, and in Ghezzi, et al., J Am Soc Nephrol. 2017 March ; 28 (3):802-810.

Agents include an inhibitor of SGLT2. In some embodiments, the inhibitor is a gliflozin. In some embodiments, the gliflozin is dapagliflozin, canagliflozin, empagliflozin, or ertugliflozin. Agents can be provided in the form of a composition suitable for administration to a subject in accordance with the methods described here.

The kit of the invention will typically comprise the container(s) described above and one or more other containers comprising materials desirable from a commercial and user standpoint, including buffers, diluents, filters, needles, syringes, and package inserts with instructions for use. In addition, a label can be provided on the container to indicate that the composition is used for a specific application, and can also indicate directions for use, such as those described herein. Directions and or other information can also be included on an insert, which is included with the kit.

### EXAMPLES

The following examples are presented to illustrate the present invention and to assist one of ordinary skill in making and using the same. The examples are not intended in any way to otherwise limit the scope of the invention.

### Example 1

### Sodium-Glucose Transporter 2 is a Diagnostic and Therapeutic Target for Early-Stage Lung Adenocarcinoma

This Example demonstrates that the glucose transporter, sodium-glucose transporter 2, is not only expressed in lung cancer but also specifically found in early-stage tumors. Using a radiolabeled tracer specific for this receptor, positron emission tomography was performed to identify early tumors that would otherwise remain undetectable. In addition, the results show that a class of diabetes drugs called gliflozins, which target the same receptor, is effective at targeting these lung tumors in mouse models. The results of this study have been published at Scafoglio et al., 2018, Science translational medicine, 10 (467). Figures and other details related to this study can be found in this 2018 publication.

The diagnostic definition of indeterminate lung nodules as malignant or benign poses a major challenge for clinicians. We discovered a potential marker, the sodium-dependent glucose transporter 2 (SGLT2), whose activity identified metabolically active lung premalignancy and early-stage lung adenocarcinoma (LADC). We found that SGLT2 is expressed early in lung tumorigenesis and is found specifically in premalignant lesions and well-differentiated adenocarcinomas. SGLT2 activity could be detected in vivo by positron emission tomography (PET) with the tracer methyl 4-deoxy-4-[18F] fluoro-alpha-D-glucopyranoside (Me4FDG), which specifically detects SGLT activity. Using a combination of immunohistochemistry and Me4FDG PET, we identified high expression and functional activity of SGLT2 in lung premalignancy and early-stage/low-grade LADC. Furthermore, selective targeting of SGLT2 with FDA-approved small-molecule inhibitors, the gliflozins, greatly reduced tumor growth and prolonged survival in autochthonous mouse models and patient-derived xenografts of LADC. Targeting SGLT2 in lung tumors may intercept lung cancer progression at early stages of development by pairing Me4FDG PET imaging with therapy using SGLT2 inhibitors.

### Study Design

The purpose of this study was to evaluate SGLT2 as a diagnostic and therapeutic target for early-stage lung cancer. We validated this by IHC in human LADC specimens and by PET imaging and therapeutic trials in mouse models. For the IHC in human specimens, the purpose of the analysis was to assess the correlation between SGLT2 expression and tumor grade and stage. Samples of LADC were retrospectively selected from the UCLA lung tumor bank according to the pathologic grade and stage. The quantification of the signal was performed blindly by a board-certified pathologist using the Aperio ImageScope software. For the imaging and therapeutic trials in mouse models, we used a Kras^{G12D}-driven, p53-null GEMM and PDXs of human LADC in nonobese diabetic (NOD), severe combined immunodeficiency (SCID), interleukin-2 receptor gamma knockout (NSG) mice. Mice were stratified to make the treatment groups comparable for mouse age (22.3γ0.54 weeks), sex (63% female, 47% male), body weight (33.5±0.66 g), and tumor burden (estimated by bioluminescence signal in the GEMMs and by volumetric determinations from CT scans in PDXs). To determine the group size, we used a GEE model (54) to compare tumor size curves over time, assuming a two-sided 0.05 level of significance. With 12 mice per group, we calculated 86% power; therefore, for our therapeutic trials, we used groups of at least 12 mice. The tumor burden in the experimental groups was evaluated by objective measurements: (i) for the GEMMs, weekly bioluminescence measurements throughout the study: (ii) for the PDXs, measurement of tumor volumes from CT scans, performed by blindly designing ROIs encompassing the whole tumor volume; and (iii) measurement of tumor area by Definiens software in histologic lung sections stained with H&E.

### Mouse Models

All experiments performed in mice were approved by the UCLA Institutional Animal Care and Use Committee and were carried out according to the guidelines of the Department of Laboratory Animal Medicine at UCLA. All mice were housed in pathogen-free facilities at UCLA.

Genetically engineered murine model. For our imaging and therapeutic trials in GEMMs, we used Lox-Stop-Lox Kras^{G12D}, p53^{lox/lox}, Rosa26-Lox-Stop-Lox-Luc mice previously established in our laboratory, inbred on a Friend virus B (FVB) background. The mice were bred in our colony at UCLA. Lung tumors were induced by intranasal administration of Adeno-Cre (purchased from University of Iowa Viral Vector Core) as previously described (30). After tumor induction, the tumor burden was estimated by weekly bioluminescence performed on an IVIS Spectrum In Vivo Imaging System (PerkinElmer) 10 min after intraperitoneal injection of luciferin (150 mg/kg).

Patient-derived xenografts. The tumor samples were obtained from surgically resected specimens after informed consent under the UCLA Lung SPORE (PDX #004 and #011) or the Long Beach Memorial Hospital Institutional Review Board protocol (PDX #013). One of the PDXs (#186) was purchased from the Jackson laboratories. PDXs were established and passaged in NSG mice. NSG mice were procured from the UCLA Radiation Oncology breeding colony. For each mouse, two small pieces of tumor tissue (4 mm³) were implanted subcutaneously in the flank regions.

### MicroPET Imaging

For the PET imaging experiment in the GEMM, the mice were scanned 12 weeks after the Adeno-Cre inhalation (1:200 dilution). For the time-course imaging, the mice received a much lower dilution of Adeno-Cre (1:10,000), and the mice were imaged when the average lung nodule maximum diameters were about 7 mm. For the microPET in PDXs, a subset of the mice (PDXs #004, #013, and #186) received both Me4FDG and FDG PET scans the day before and 2 weeks after the beginning of treatment to evaluate the response of glucose transporter activity to the treatment.

The animals were anesthetized with 1.5% (v/v) isoflurane in oxygen, were given a dose of 100 μCi of Me4FDG or FDG via tail vein injection, and were maintained under anesthesia for 1 hour of unconscious uptake. The mice were then immobilized on the imaging bed and received a 10-min static PET scan followed by a CT scan. Each mouse received two different PET scans with the two tracers (Me4FDG and FDG) on consecutive days to allow for tracer decay. The equipments used were Focus 220 microPET scanner (Concorde Microsystems) and Inveon microPET scanner (Siemens) for the microPET scans and CrumpCAT (UCLA Crump Institute) for the microCT.

The PET data were analyzed with AMIDE software version 1.0.4 (amide.sourceforge.net/) (55). ROIs for the measurement of tumor uptake were drawn corresponding with single lung nodules as identified by CT images. For the GEMMs, which typically present with small intrathoracic nodules with regular shape, ellipsoid ROIs were considered to be an acceptable approximation of tumor volume. For advanced lung nodules that did not have perfectly ellipsoid shape and smooth borders, the ROIs were placed in the center of the tumor nodule to include as much tumor volume as possible inside the ROI. To compare FDG and Me4FDG uptake, the same ROIs were used in the same mice scanned with the two different tracers, such that comparable tumor volumes were measured with the two tracers. For the PDXs, which typically show larger and irregularly shaped tumors. isocontour ROIs were designed on the basis of the CT scans to encompass the whole tumor volumes. The percentage of injected dose for each ROI was calculated by dividing the measured activity in the ROI by the total injected dose, as measured from the PET image by designing an ROI encompassing the whole mouse. The analysis of signal-to-noise ratio in the mouse nodules was performed as described in (31). Briefly, we evaluated the background signal for each mouse by designing a ROI corresponding to the normal lung. We then calculated the CNR for each nodule by using the following formula (31)

$$CNR = {C}_{1} \times \sqrt{{n}_{l}} \times {SNR}_{pixel} \qquad ( 1 )$$

where Cₗ is the lesion to background contrast, nₗ is the number of pixels in the ROI, and SNRₚᵢₓₑₗ is the signal-to-noise ratio for a single pixel in the background

$${C}_{t} = \frac{{S}_{t} - {S}_{b}}{{S}_{b}} and {SNR}_{pixel} = \frac{{S}_{b}}{{σ}_{b}} \qquad ( 2 )$$

The smallest lesion activity that can generate a CNR greater than 3 to 5 is called the minimum detectable activity. For the purpose of this analysis, we considered a CNR ≥4 as the specific signal.

### Therapeutic Studies in Mice

Genetically engineered mouse model. Tumors were induced in KP_{luc} mice by inhalation of Adeno-Cre (1:10 dilution). Two independent trials were performed starting 2 weeks after tumor induction. In the first trial (n=12 mice per group), mice were treated for 6 weeks and then sacrificed for lung collection and fixation in formalin. In the second trial (n=15 per group), mice were treated until death for survival analysis. The two therapeutic groups were as follows: (i) placebo, receiving daily oral gavage with vehicle (0.5% hydroxypropyl-methyl cellulose); and (ii) canagliflozin, receiving a daily dose of canagliflozin (30 mg/kg via oral gavage), as previously described (25). In the survival cohort, two mice (#3416 and #3418) were censored on days 45 and 57, respectively, for esophageal rupture because of complication of the oral gavage.

Patient-derived xenografts. The mice were randomized in two therapeutic groups (same as for the trials in GEMMs): placebo and canagliflozin (30 mg/kg per day) The number of mice per group was four for PDX #011, eight for PDX #013, three for PDX #004, and five for PDX #186; each mouse was inoculated with two tumors (one tumor on each flank). Some of the tumors of PDX #011 developed soft tissue metastases in the axillary regions, and these were counted as separate tumors. Overall, 38 tumors were included in the placebo and 39 tumors in the canagliflozin group. The mice were treated for 1 month and then sacrificed, and the tumors were collected for histology and IHC. The mice of PDX #011 were treated only for 2 weeks because extremely rapid tumor growth in the placebo group required premature sacrifice of the animals. The tumor volumes at 2 weeks were counted as final tumor volumes.

### Immunohistochemistry

For the GEMMs, the mouse lungs were collected and inflated with 10% formalin in phosphate-buffered saline and then incubated in formalin for 24 hours. For the PDXs. subcutaneous tumors were collected and incubated in formalin for 24 hours. All tissues were paraffin-embedded and sliced into 4-μm sections in the Translational Pathology Core Laboratory (TPCL) at UCLA. For human lung cancer samples, tissue blocks were obtained anonymously from the UCLA Lung SPORE tissue bank and from the Long Beach Memorial Hospital.

For IHC staining, the slides were deparaffinized by overnight incubation at 65° C., followed by rehydration by serial passages in xylenes (three washes of 5 min in 100% xylenes) and decreasing concentrations of ethanol (two washes in 100% ethanol, two washes in 95%, one wash in 80%, one wash in 70%, and one wash in water). Antigen retrieval was performed for 20 min in 10 mM tris-HCl and 1 mM EDTA (pH 8.0) for SGLT2 and GLUT1 antibodies and in 10 mM citrate (pH 6.0) for Ki67. Blocking was performed with 5% goat serum for 1 hour at room temperature, followed by incubation with primary antibodies overnight at 4° C. Incubation with biotin-labeled secondary antibody was performed at room temperature for 1 hour, followed by incubation with avidin-biotin peroxidase complex (ABC, Vector Laboratories) and ImmPACT 3,3′-diaminobenzidine (Vector Laboratories) for 1 min. Counterstain was performed with Harris' hematoxylin diluted 1:5 in water. For SGLT2, two different antibodies were used: Abcam ab85626 (1:1000) for mouse tissues and Novus Biologicals NBP1-92384 (1:250) for human and mouse tissues. The antigenic peptide for the Novus antibody is FHEVGGYSGLFDKYLGAATSLTVSEDPAV GNISSFCYRPRPDSYHLL (SEQ ID NO: 1); for the Abcam antibody, the sequence is proprietary but included in residues 250 to 350 of human SGLT2. For GLUT1, the Alpha Diagnostics GT11A antibody (1:200) was used. For Ki67, the Thermo Fisher Scientific SP6 antibody (1:200) was used.

After the staining, digital images of the slides were obtained with an Aperio ScanScope slide scanner (Leica Biosystems). For the mouse tissues, the IHC signal was quantified with the Definiens Tissue Studio software. For the human tissues, the quantification was performed blindly by a board-certified pathologist using the Aperio ImageScope software.

### Statistical Analyses

The association between SGLT2 expression and Me4FDG uptake in mouse tissue was assessed using a GEE model (54) to accurately account for the same mouse being measured multiple times (multiple tumors per mouse or same mouse over time). We ran a similar model for testing the association between SGLT2 and GLUT1 expression. The associations were also quantified using Pearson's correlation coefficient. The association between time (weeks 2, 5, and 8) and SGLT2 (or GLUT1) was formally assessed using the Jonckheere-Terpstra test for ordered alternatives.

For the human samples, SGLT2 expression was computed using a weighted average, where each staining score assigned by the Aperio software (3+, very strong; 2+, strong: 2+, light; 1+, very light; 0, no signal) was multiplied by the corresponding percentage of cells in each sample. We then assessed the directional association between morphology categories (lepidic, moderately differentiated, and poorly differentiated) and SGLT2 or GLUT1 score using the Jonckheere-Terpstra test. Statistical analyses were performed using the Statistical Package for the Social Sciences (SPSS) v24.

For the therapeutic trials in the GEMMs, tumor growth curves (slopes) were compared between canagliflozin and control groups using GEE models (54), with terms for time, group, and the time by group interaction. These models also included a random effect for mouse to account for the repeated observations of tumor size over time. We used log transformations to normalize the tumor size outcomes. For the therapeutic trials in PDXs, a linear mixed-effects model for log tumor volume was used, with terms for fixed effects (treatment group) and random effects (PDX trial; mice within trial clustered random effect: 38 to 39 distinct tumors in 20 distinct mice). The data analysis was performed using PROC MIXED from the Statistical Analysis Software (SAS) v9.4. All other group comparisons were performed using the two-sample t test unless otherwise noted. P values <0.05 were considered statistically significant throughout

### Results

SGLT2 is a glucose transporter specifically expressed in lung premalignancy and well-differentiated cancer

We have previously shown the expression of SGLT2 in human pancreatic and prostate adenocarcinomas (25) and in glioblastomas (28). Furthermore, SGLT2 expression has been observed in metastatic lung cancers (29). To investigate glucose transporter expression in LADC, we performed immunohistochemistry (IHC) staining for SGLT2 and GLUT1 proteins on 58 human LADC samples (stage I to stage IV). The specificity of the SGLT2 antibody was confirmed by IHC in knockout mouse kidneys lacking the SGLT2 protein and by preincubation of the antibody with the antigenic peptide. The clinical and pathological features of the patients are presented in table S1 (see online publication), along with the quantification of SGLT2 and GLUT1 staining by IHC. SGLT2 was highly expressed in well-differentiated (lepidic) and moderately differentiated LADC; its expression was reduced in poorly differentiated, solid-growth disease. Conversely, GLUT1 expression was low in well-differentiated LADC and progressively increased in poorly differentiated tumors. Quantification of the IHC signal showed a significant reduction of SGLT2 (P=0.001) and an increase in GLUT1 (P<0.001) staining as the tumor progressed from lepidic (well differentiated) to moderately and poorly differentiated LADC. We therefore hypothesized that early-stage LADCs rely on SGLT2 for glucose uptake, whereas more advanced tumors rely on GLUT1. We did not observe a significant change in SGLT2 and GLUT1 staining from stage I to stage IV cancers, but samples from stage IA patients had significantly higher expression of SGLT2 (P=0.04) and lower expression of GLUT1 (P=0.02) than samples from all other stages. This prompted us to hypothesize that SGLT2 expression is an early event in lung cancer development. We investigated SGLT2 and GLUT1 expression in premalignant lung lesions observed in 9 of the 58 patients, whose clinical characteristics are reported in Table 1. In most patients, multiple GGNs were discovered incidentally on CT performed for other reasons, on screening CT, or as synchronous lesions at the time of resection of an invasive LADC (Table 1). We performed IHC on 11 AAH lesions, 3 AIS lesions, and 3 MIA lesions. SGLT2 was not expressed in normal lung alveoli but was up-regulated in premalignant lesions. GLUT1 expression was absent in normal alveoli and premalignant lesions.

TABLE 1
Clinical characteristics of the patients
with AAH included in the study.

| Pa- | | | | | | Patho- | Over- |
|---|---|---|---|---|---|---|---|
| tient | | | Smoking | Pack | Means of | logical | all |
| # | Age | Sex | status | years | detection | stage | stage |
| 1 | 58 | M | Current | 40 | Incidental | T1aN0M0 | IA |
| 2 | 59 | M | Former | 60 | Symptomatic | T3N0M0 | IIB |
| 3 | 54 | F | Never | 0 | Symptomatic | TisN0M0 | 0 |
| 4 | 73 | F | Former | 31 | Screening | T1aN0M0 | IA |
| | | | | | chest X-ray | | |
| 5 | 82 | F | Never | 0 | Incidental | T1aN0M0 | IA |
| 6 | 75 | F | Former | 20 | Incidental | T3N0M0 | IIB |
| 7 | | | | | | | |
| 8 | 77 | M | | | | T1bN0M0 | IA |
| 9 | 70 | F | Former | 28 | Surveillance | T3N0M0 | IIB |

F, female; M, male.

The pattern of IHC staining with SGLT2 in low-grade and GLUT1 in high-grade LADC was confirmed in four PDXs. PDX #004 was characterized as a predominantly well-differentiated LADC that expressed high SGLT2 and had no GLUT1 expression. In PDX #011 and PDX #186, the predominant histology was moderately differentiated LADC that expressed both SGLT2 and GLUT1. Last, PDX #013 was characterized as predominantly poorly differentiated LADC that was positive for GLUT1 but negative for SGLT2. Together. these data suggested that SGLT2 is predominantly expressed in human premalignancy and early-stage, well-differentiated LADCs, and as lung tumors progress to advanced and poorly differentiated cancers, they up-regulate GLUT1 as the dominant transporter.

PET imaging reveals differential activity of SGLT2 and GLUT1 transporters in LADC

We have previously profiled functional expression of SGLT2 in mouse xenografts of pancreatic, prostate, and brain tumors and have shown that PET imaging with FDG alone fails to comprehensively profile glucose uptake in tumors that express SGLT2 (25, 28). To study the functional activity of SGLTs in LADC, we used a conditionally activated Lox-Stop-Lox KrasG12D, p53lox/lox, Rosa26-Lox-Stop-Lox-Luc (referred to as KPluc) GEMM, in which tumor induction with adenovirus expressing Cre recombinase simultaneously activates oncogenic KrasG12D, deletes p53, and induces activation of a luciferase reporter enabling bioluminescent imaging of tumors (30). In this model, LADCs are induced by inhalation of adenovirus encoding for Cre recombinase. To estimate the activity of GLUTs and SGLTs in LADC, we performed PET imaging with FDG and Me4FDG in KPluc mice with advanced-stage tumors. The same mice were imaged with both tracers on different days. We observed a modest but significant correlation between FDG and Me4FDG uptake in lung tumors [Pearson's coefficient (r)=0.4966, P<0.0001], suggesting that advanced carcinomas may use both SGLT2 and GLUT1 for glucose transport. A closer observation identified a subpopulation of smaller Me4FDG-positive lesions in KPluc mice that were negative for FDG uptake Representative PET/CT scans of a mouse with multiple lung nodules included a tumor that was dually positive for Me4FDG and FDG, as well as two smaller nodules (see online manuscript for corresponding images). Using three-dimensional (3D) rendering of the regions of interest (ROIs) to locate the exact position of the tumor nodules in the lungs, we correlated the PET signal with tumor histology and IHC. The larger tumor, which was positive for both FDG and Me4FDG uptake, expressed both SGLT2 and GLUT1. Histological analysis for the smaller Me4FDG-positive tumor confirmed high SGLT2 and low GLUT1 expression.

We evaluated the minimum detection limit of Me4FDG to detect early-stage lung nodules by calculating the contrast-to-noise ratio (CNR) as described in (31). The lung background was evaluated by designing ROIs corresponding to the normal lungs. The formulas used for calculating the CNR are described in Materials and Methods. We used a stringent cutoff, setting the minimum detection level at lesions with a CNR≥4. This analysis showed that CNR for Me4FDG is linearly related to lesion size, as expected. Tumors 12 and t3 had CNRs of 4.47 and 7.83, respectively. The smallest lesion with a CNR≥4 for Me4FDG was t2, which measured 0.52 mm3 in volume (diameter, 1.0 mm). IHC staining of this lesion showed SGLT2 positivity in 71% of the cells. Overall, the quantification of the IHC signal showed that SGLT2 expression correlated with Me4FDG, as expected. Likewise, GLUT1 expression is correlated with FDG uptake (22, 32-34).

The positive correlation between Me4FDG and FDG was not expected, considering the reciprocal pattern of SGLT2 and GLUT1 expression observed in the human specimens. However, upon closer inspection of nodules expressing both SGLT2 and GLUT1. we observed that most of the advanced carcinomas expressed both transporters, but the two proteins usually did not colocalize in the same cells. Rather, they were observed in different regions of the tumor nodules, highlighting the intratumor heterogeneity of glucose uptake. Specifically, we noticed that SGLT2 was expressed in well-differentiated areas of the tumor, whereas GLUT1 was more abundant in poorly differentiated regions, confirming the pattern already observed in human LADCs.

We therefore measured the IHC signal in subareas of the tumor nodules that could be clearly identified in the H&E staining as purely low grade or high grade. Similarly to human LADO, the lung tumors in KPluc mice show a very heterogeneous morphology, with well- and moderately differentiated areas coexisting with poorly differentiated cell clusters. In many cases, poorly differentiated cells are interspersed within well- and moderately differentiated cells, in a mosaic pattern. We therefore quantified SGLT2 and GLUT1 expression only in the ROIs corresponding to clearly discernible areas of the tumors that were purely low grade (well or moderately differentiated) or high grade (poorly differentiated). This analysis showed that SGLT2 and GLUT1 were negatively correlated We color coded the ROIs to facilitate distinction between those corresponding to high-grade tumor regions and those corresponding to low-grade tumor regions. The high-grade ROIs clustered with a GLUT1-positive signal, whereas low-grade ROIs clustered with a SGLT2-positive signal.

We next quantified SGLT2 and GLUT1 staining in lung tumors from KPluc mice. IHC staining revealed, for example, adjacent sections of a sample stained with antibodies against SGLT2 and GLUT1, that could be compared with the corresponding H&E staining. One area in such a representative sample, is well differentiated and has high SGLT2 and low GLUT1 expression, while a nearby region is poorly differentiated and shows low SGLT2 and high GLUT1 signal. Quantification of signal intensity in all lung tumors from n=7 mice showed a significantly higher SGLT2 staining in low-grade areas of the tumors (P<0.001) and higher GLUT1 signal in high-grade areas (P<0.001). This heterogeneity of glucose transport mirrors the heterogeneity observed in human tumors. Human LADCs often present with a mix of well- to poorly differentiated tumor cells. We found several instances in which different areas of the same tumor stained with either SGLT2 or GLUT1 in a mutually exclusive pattern. SGLT2 staining was confined to well- and moderately differentiated areas, whereas GLUT1 staining was localized in poorly differentiated regions of the same tumor. Together, these data show that SGLT2 and GLUT1 represent two independent mechanisms of glucose uptake in cancer cells, with SGLT2 prevalent in low-grade LADC and GLUT1 prevalent in high-grade LADC.

### Premalignant and Early LADCs Predominantly use SGLT2 to Transport Glucose into Tumors

Our data suggested that Me4FDG may detect early LADC lesions that are FDG negative yet metabolically active. They also supported the premise that SGLT2 expression is an early event in LADC oncogenic transformation.

To identify the stage of lung carcinogenesis in which SGLT2 is first up-regulated, we performed serial IHC in KPluc GEMMs 2, 5, and 8 weeks after tumor induction. Premalignant AAH lesions developed in these mice as early as 2 weeks after tumor induction. By week 5, small adenomas were observed, and by week 8, the mice had advanced adenocarcinomas. IHC showed that SGLT2 expression was highest in premalignant lepidic lesions at week 2 and progressively diminished in early adenomas and in advanced LADC. Conversely, GLUT1 expression was not detectable in early lesions at week 2 but became progressively stronger as the tumors progressed to adenomas and advanced LADC, suggesting an evolution in the pattern of expression of glucose transporters, with SGLT2 exclusively expressed in early stages and both SGLT2 and GLUT1 present in a mosaic pattern in late stages of lung carcinogenesis. Quantification of the signal confirmed a significant decrease in SGLT2 (P<0.001) and increase in GLUT1 (P=0.048) as the tumors became more advanced.

To better characterize the evolution of SGLT2 and GLUT1 activity during lung tumorigenesis, we performed a time course of repeat Me4FDG and FDG PET imaging on KPluc mice. The first PET scan was performed when lung nodules reached an average size of 7 mm3, followed by two biweekly scans with a total of three time points. Analysis of tracer uptake at different time points showed that Me4FDG was taken up in early lesions, which were mostly negative for FDG. As the tumors progressed and became more advanced, they increased their uptake of FDG, whereas Me4FDG uptake remained constant. Quantification of tracer uptake in all the lung nodules included in the study (n=16 tumors in seven mice) confirmed that early tumors had high Me4FDG and low FDG uptake, whereas late tumors were more heterogeneous, with a slightly reduced Me4FDG uptake and significant up-regulation of FDG uptake (P<0.001). These results are consistent with the expression of both transporters in advanced tumors and with the positive correlation between Me4FDG and FDG uptake observed in advanced tumors.

Together, these data show that early-stage LADCs and premalignant lesions express SGLT2 and use only sodium-dependent transport for glucose uptake, whereas more advanced lesions up-regulate GLUT1 expression. These results suggest a complex and spatially heterogeneous pattern of glucose uptake that engages both SGLT- and GLUT-dependent glucose transport during LADC progression.

### Gliflozins Suppress Growth of Early Stage LADC and Extend Survival in KPluc GEMMs

Because we observed only SGLT-dependent glucose uptake in early-stage lung nodules, we hypothesized that SGLT2 is required for tumor growth in the early stages of lung carcinogenesis. Therefore, we reasoned that pharmacological inhibition of SGLT2 would slow tumor development in KPluc GEMMs and extend overall survival. Gliflozins are U.S. Food and Drug Administration (FDA)-approved drugs for the treatment of diabetes (35, 36) and have been tested against pancreatic cancer in xenografts (25). Gliflozins function by inhibiting glucose reabsorption in the kidneys, resulting in significant (P<0.01) excretion of Me4FDG in the urine. We first confirmed that gliflozins were selective inhibitors of SGLT2 activity by performing Me4FDG PET imaging on KPluc mice, with or without coadministration of a single intravenous dose of the SGLT2 inhibitor dapagliflozin (25, 37). As anticipated (25, 38), dapagliflozin reduced Me4FDG uptake in the tumors and in the heart, liver, and skeletal muscle.

We then performed preclinical studies on KPluc mice to test the SGLT2 inhibitor canagliflozin, which was recently approved by the FDA for diabetes and reported to be a more effective SGLT2 inhibitor than dapagliflozin (39). We started treatment with canagliflozin (30 mg/kg per day by oral gavage) (25) 2 weeks after tumor induction, when only premalignant lesions were present. We performed two experiments: In the first trial, we treated mice for 6 weeks, followed by sacrifice and histological analysis of lungs and tumors (n=12 mice per group); in the second trial, we treated mice for up to 3 months for survival analysis (n=15 mice per group). Tumor burden, estimated by bioluminescence imaging (BLI), was significantly lower (P<0.001) in the treatment than the placebo group Quantification of the BLI signal for both treatment groups up to week 8 showed that canagliflozin significantly delayed the onset of tumors compared to the placebo group (P<0.001). In addition, plotting the BLI for individual animals up to the end of the trial at week 14 showed that the BLI signal remained lower in the treatment group as compared to the placebo group. Histological analysis of the lungs and measurement of tumor area confirmed that canagliflozin-treated mice had a significantly lower tumor burden than in the placebo group (P<0.05), both at 8 weeks and in the survival cohort. A Ki67 stain of the mouse tumors showed a significantly lower (P=0.001) proliferation rate in the canagliflozin treatment group compared with that in the placebo group but only in premalignant lesions and not in solid LADCs, suggesting that SGLT2 inhibition may target premalignant lesions with higher efficacy than advanced carcinomas. This is consistent with the observation that SGLT2 is expressed predominantly in early lesions.

Overall, canagliflozin treatment significantly prolonged mouse survival (P=0.0003) compared with the placebo group. However, tumors eventually escaped SGLT2 inhibition and formed large tumor masses even within the canagliflozin group. We analyzed SGLT2 expression in the lungs of mice from both treatment groups and showed that SGLT2 expression was low in the placebo group, with only 17% of tumor cells staining positive for SGLT2, consistently with the advanced nature of these tumors. Tumors in the canagliflozin group had significantly higher SGLT2 expression (P<0.0001): 30% of the tumor cells had positive SGLT2 staining. This suggests that overexpression of the transporter may be a compensatory mechanism induced by SGLT2 inhibition. Together, these data suggest that SGLT2-mediated glucose transport is an early requirement for lung carcinogenesis and may sustain the increased proliferation rate associated with progression from premalignancy to invasive cancer and tumor growth.

### Me4FDG PET-Guided Inhibition of SGLT2 Activity in LADC Reduces Tumor Growth in PDXs

We next performed a therapeutic trial with canagliflozin (30 mg/kg per day by oral gavage for 1 month) (25) in the same four PDXs to test the relevance of targeted SGLT2 inhibition in human cancers. Mice were imaged by Me4FDG and FDG PET/CT before treatment and 2 weeks after starting treatment. Weekly CT scans were acquired to monitor tumor burden. The mice were sacrificed, and the tumors were analyzed after 1 month of treatment. Overall, we observed a significant (P=0.003) reduction in tumor volumes in the treatment group compared with the placebo group. Treated mice had final tumor volumes about 47% lower than the control mice (geometric mean ratio, 0.53; 95% confidence interval, 0.36 to 0.80; P=0.003), after adjusting for trial and mouse random effects.

Despite the variability in SGLT2 staining between moderately and poorly differentiated PDXs, all four PDXs displayed a detectable uptake of Me4FDG and a similar response to SGLT2 inhibition. This was expected, considering that these PDXs were not purely SGLT2 positive or SGLT2 negative, but in each PDX well and moderately differentiated, SGLT2-positive areas coexisted with poorly differentiated, SGLT2-negative areas of the same tumor.

Overall, Me4FDG uptake increased from day 0 to day 14 in the placebo group, whereas canagliflozin significantly reduced Me4FDG uptake (P<0.001), as expected. Me4FDG uptake before treatment in the placebo group significantly correlated with tumor volume fold change from the beginning to the end of the trial (P<0.001), confirming an important role of SGLT2-dependent glucose uptake in the regulation of tumor growth. In the canagliflozin group, Me4FDG scans before treatment showed no significant correlation with tumor volume fold change. Moreover, the percent reduction of Me4FDG uptake observed after canagliflozin treatment negatively correlated with the fold increase in tumor volume (P=0.027), confirming that a reduction in Me4FDG uptake after treatment can be used as a marker of response to SGLT2 inhibition. In contrast, FDG signal did not significantly change from day 0 to day 14 in either the placebo or the canagliflozin group. In addition, pretreatment uptake or changes in FDG uptake between pre- and posttreatment did not correlate with the fold change in tumor volume. These data support the premise that SGLT2 inhibition may be effective in retarding the growth of early lesions that rely on SGLT2 for glucose uptake and that Me4FDG may be used to evaluate the response to therapeutic inhibition of SGLT2.

This example thus presents evidence that SGLT2 is a glucose transport mechanism specifically active in pulmonary premalignant lesions and in early LADC. We show the expression of SGLT2 protein by IHC with specific antibodies in human LADC specimens, in genetically engineered murine models and in PDXs of LADC. We also report SGLT2-dependent uptake of the PET tracer Me4FDG in vivo both in genetically engineered and in patient-derived murine models. Last, we confirm that SGLT2 inhibitors delay LADC development and growth in murine models, suggesting SGLT2 inhibition as a potential therapeutic strategy for premalignant and early-stage LADC. These data highlight the importance of SGLT in early stages of LADC development, both in human tumors and in murine models.

These data suggest that there is a progression of glucose transporter expression during LADO carcinogenesis: Early-stage lesions express only SGLT2, whereas more advanced lesions display a spatially complex and heterogeneous pattern of SGLT2 and GLUT1 expression. Consistently, early-stage tumors accumulate only Me4FDG and not FDG, whereas advanced tumors take up both tracers. SGLT2 is most prominent in well-differentiated tumor cells, whereas GLUT1 becomes prevalent in poorly differentiated tumor cells. The mechanisms that drive this evolution in glucose transport in cancer are unknown and will need to be the subject of future investigations. However, we hypothesize that hypoxia is a major driver of the transition from SGLT2- to GLUT1-dependent glucose uptake in LADC. Hypoxia-inducible factor 1α up-regulates GLUT1 expression (40-42). We therefore speculate that premalignant and early-stage lesions, which are well oxygenated and perfused, preferentially express SGLT2. In contrast, it may be that, in advanced hypoxic lesions, GLUT1-mediated glucose transport dominates the tumor landscape.

The discovery of SGLT2 expression in pulmonary premalignancy and early LADC has important diagnostic and therapeutic applications. The National Lung Screening Trial (NLST) has shown that low-dose helical CT in older, high-risk smokers can reduce lung cancer-related mortality by 20% compared with chest x-ray (43). However, CT imaging lacks specificity in distinguishing benign from malignant solitary pulmonary nodules (44). Moreover, high-resolution CT has increased the detection rates of indeterminate lung lesions (45), including both benign lesions and premalignant or early adenocarcinomas (46-48). which require additional imaging or invasive procedures for diagnosis. FDG PET has proven to be ineffective in identifying premalignancy or early LADC (17, 23), particularly in the setting of subsolid nodules (15-17) such as AAH, AIS, or MIA lesions (3, 14). Patients in the NLST with benign lesions (73%) received invasive diagnostic procedures (49). In addition to the costs and potential complications associated with these procedures, a quality-of-life (QOL) study on the impact of low-dose CT (LDCT) screening found that 46% of patients reported psychological distress while awaiting imaging results (50). This is particularly notable when considering that 16% of lesions identified on baseline screening LDCT in the NLST were ground-glass opacities that demonstrated relatively constant annual rates of lung cancer diagnosis over the course of 6 years of follow-up. Subsolid lung nodules commonly represent the early spectrum of LADC and may persist for years before malignant progression. Improved diagnostic tools could potentially reduce cost, invasive procedures, and radiation exposure, as well as improve QOL for patients with indeterminate lung nodules requiring surveillance. In addition to GGNs identified incidentally or by screening, it is common to detect multiple GGNs in patients who undergo surgery for an invasive LADC: Me4FDG PET could aid the follow-up of these lesions and help predict the risk of progression to invasive cancer.

This example presents evidence that, in premalignant and early lepidic lesions, cellular glucose uptake occurs via SGLT2 rather than GLUT1 to satisfy metabolic demands. Therefore, it is anticipated that measurement of SGLT-mediated glucose utilization with the tracer Me4FDG will be valuable in classifying subsolid nodules as benign disease or early lesions within the spectrum of adenocarcinoma. This has the potential to answer an unmet need in the field of lung cancer that CT and FDG PET alone cannot address. The next goal is to extend this work to clinical trials to evaluate Me4FDG for the diagnostic characterization of indeterminate lung nodules and GGNs and for predicting the clinical behavior of these lesions.

We have also shown that SGLT2 inhibitors hinder tumor progression by limiting glucose supply in cancer cells and that Me4FDG can be used to evaluate the response of LADCs to SGLT2 inhibition by PET imaging before and after receiving treatment. Specific SGLT2 inhibitors (gliflozins), which are FDA approved for the treatment of diabetes, function by lowering the renal threshold for glucose reabsorption and therefore induce glycosuria and reduce blood glucose in patients with diabetes (35, 36). We have previously shown that gliflozins have antitumor activity against pancreatic tumors in a xenograft model (25). Here, we found that gliflozins specifically target lung premalignancy, effectively reduce tumor burden, and prolong survival if administered at an early stage. Our studies in PDX models treated with canagliflozin showed that Me4FDG uptake before treatment correlated with tumor volume fold decrease after treatment. This suggests that Me4FDG PET imaging could help identify individuals with premalignancy or early LADC with active SGLT2 transporters. In this context, gliflozin therapy could be applied as a cancer interception strategy to prevent or delay malignant progression of subsolid lesions detected by Me4FDG PET and CT (8, 9). This strategy would serve patients with other tobacco-associated cardiopulmonary diseases who are poor candidates for surgical resection. The ability to reliably detect premalignant and early stage LADCs with the use of Me4FDG PET could enable more timely interventions, interrupting the progression to invasive, more advanced disease and thus improving long-term outcomes.

We further observed that Me4FDG PET, by detecting SGLT-dependent glucose transport in vivo, can be used to assess response to treatment with SGLT2 inhibitors. In particular, measurement of Me4FDG uptake in PDXs before and after beginning gliflozin treatment allowed us to establish an inverse correlation between the reduction in Me4FDG uptake as a consequence of the treatment and the rate of tumor volume increase. In patients with FDG-avid tumors, monitoring metabolic responses to drug therapy can be relevant for prognostic assessment and clinical decision making regarding treatment (51-53). Me4FDG PET in mice showed similar if not better minimal detection limits in early-stage lesions compared to FDG PET and would be expected to perform in an equivalent manner in humans (28). Therefore, we anticipate that PET measurement of SGLT activity in lung premalignancy and adenocarcinomas before and after the beginning of treatment will provide an invaluable precision medicine tool to evaluate the response of premalignant lesions to SGLT2 inhibitors.

This study has some limitations. First, we were not able to demonstrate a significant correlation between SGLT2 expression and tumor stage. This is probably because of the low number of samples, especially considering that most surgical samples in the University of California, Los Angeles (UCLA) tumor bank were from early-stage tumors, with a relatively low number of advanced LADCs. However, we were able to find a specific profile of SGLT2 expression in lepidic and well-differentiated lesions. Second, we demonstrated Me4FDG uptake in GEMMs and in PDXs but we did not perform Me4FDG PET in patients with LADC: this will need to be the focus of future studies (see Examples 2 and 3 below). Third, we cannot exclude that the effect of SGLT2 inhibitors on tumor volume and mouse survival is because of a systemic effect of lowering blood glucose rather than to the inhibition of glucose uptake in the tumor. Investigations using a conditional knockout of SGLT2 in the KPluc model should discriminate between these two possibilities.

### REFERENCES AND NOTES

1. J. Remon, B. Besse. Curr. Opin. Oncol. 29, 97-104 (2017).
2. H. I. Assi, et al. Cancer 124, 248-261 (2018).
3. W. Weichert, A. Warth. Curr. Opin. Pulm. Med. 20, 309-316 (2014).
4. H. Kadara, et al. Respirology 17, 50-65 (2012).
5. S. Sivakumar, et al. Cancer Res. 77, 6119-6130 (2017).
6. J. C. Tsay, et al. PLOS ONE 10, e0118132 (2015).
7. A. Spira, et al. Cancer Res. 77,1510-1541 (2017).
8. A. Albini, et al. Clin. Cancer Res. 22, 4322-4327 (2016).
9. E. H. Blackburn. Cancer Prev. Res. (Phila.) 4, 787-792 (2011).
10. A. T. Ooi, et al. Cancer Prev. Res. (Phila.) 7, 487-495 (2014).
11. Y. Yatabe, et al. Lung Cancer 74, 7-11 (2011).
12. W. D. Travis, et al. J. Thorac. Oncol. 10, 1243-1260 (2015).
13. W. D. Travis, et al. Proc. Am. Thorac. Soc. 8, 381-385 (2011).
14. H. Y. Lee, et al. AJR Am. J. Roentgenol. 202, W224-W233 (2014).
15. K. Higashi, et al. J. Nucl. Med.39, 1016-1020 (1998).
16. T. W. Huang, et al. Eur. J. Surg. Oncol. 38, 1156-1160 (2012).
17. H. B. Wu, et al. Biomed. Res. Int. 2015,243681 (2015).
18. O. Warburg, Science 123, 309-314 (1956).
19. M. G. Vander Heiden, et al. Science 324, 1029-1033 (2009).
20. V. Ganapathy, et al. Pharmacol. Ther. 121, 29-40 (2009).
21. K. Adekola, et al. Curr. Opin. Oncol. 24,650-654 (2012).
22. J. Goodwin, et al. Nat. Commun. 8, 15503(2017).
23. V. Ambrosini, et al. Eur. J. Radiol. 81, 988-1001 (2012).
24. C. S. Yap, et al. Eur. J. Nucl. Med. Mol. Imaging 29, 1166-1173 (2002).
25. C. Scafoglio, et al. Proc. Natl. Acad. Sci. U.S.A. 112, E4111-E4119 (2015).
26. A. S. Yu, et al. Am. J. Physiol. Cell Physiol. 299, C1277-C1284(2010).
27. A. S. Yu, et al. Am. J. Physiol. Cell Physiol. 304, C240-C247 (2013).
28. V. Kepe, et al. J. Neurooncol 138, 557-569 (2018).
29. N. Ishikawa, et al. Jpn. J. Cancer Res. 92, 874-879 (2001).
30. D. B. Shackelford, et al. Cancer Cell 23, 143-158 (2013).
31. Q. Bao, A. F. Chatziioannou. Med. Phys. 37, 6070-6083 (2010).
32. N. Suzawa, et al. Lung Cancer 72, 191-198(2011).
33. L. F. de Geus-Oei, et al. Lung Cancer 55, 79-87 (2007).
34. M. Mamede, et al. Neoplasia 7, 369-379 (2005).
35. D. Devineni, et al. J. Clin. Pharmacol. 53, 601-610 (2013).
36. C. J. Bailey, et al. Lancet 375, 2223-2233 (2010).
37. C. Ghezzi, et al. J. Am. Soc. Nephrol. 28, 802-810 (2017).
38. M. Sala-Rabanal, et al. J. Physiol. 594, 4425-4438 (2016).
39. S. Sha, et al. Diabetes Obes. Metab. 17, 188-197 (2015).
40. D. B. Shackelford, et al. Proc. Natl. Acad. Sci. U.S.A. 106, 11137-11142 (2009).
41. C. Chen, et al. J. Biol. Chem. 276, 9519-9525 (2001).
42. H. F. Bunn, R. O. Poyton. Physiol. Rev. 76, 839-885(1996).
43. D. R. Aberle, et al. N. Engl. J. Med. 365, 395-409 (2011).
44. D. R. Aberle, et al. N. Engl. J. Med. 369, 920-931 (2013).
45. M. K. Gould, et al. Am. J. Respir. Crit. Care Med. 192, 1208-1214(2015).
46. L. C. Horn L, D. H. Johnson, Neoplasms of the Lung, in Harrison's Principles of internal Medicine, D. Kasper, A. Fauci, S. Hauser, D. Longo, J. Jameson, J. Loscalzo, Eds. (McGraw-Hill, ed. 19, 2015).
47. Y. Kobayashi, et al. Transl. Lung Cancer Res. 2, 354-363 (2013).
48. B. Chang, et al. Chest 143, 172-178 (2013).
49. P. B. Bach, et al. JAMA 307, 2418-2429 (2012).
50. K. A. van den Bergh, et al. Cancer 113, 396-404 (2008).
51. R. Takahashi, et al. Clin. Cancer Res. 18, 220-228 (2012).
52. K.-C. Ho, et al. Eur. J. Nucl. Med. Mol. Imaging 43, 2155-2165(2016).
53. J. Vansteenkiste, et al. Lancet Oncol. 5, 531-540 (2004).
54. K.-Y. Liang, S. L. Zeger. Biometrika 73, 13-22 (1986).
55. A. M. Loening. S. S. Gambhir. Mol. Imaging 2, 131-137 (2003).

### Example 2

Me4FDG Images Lesions Negative with FDG

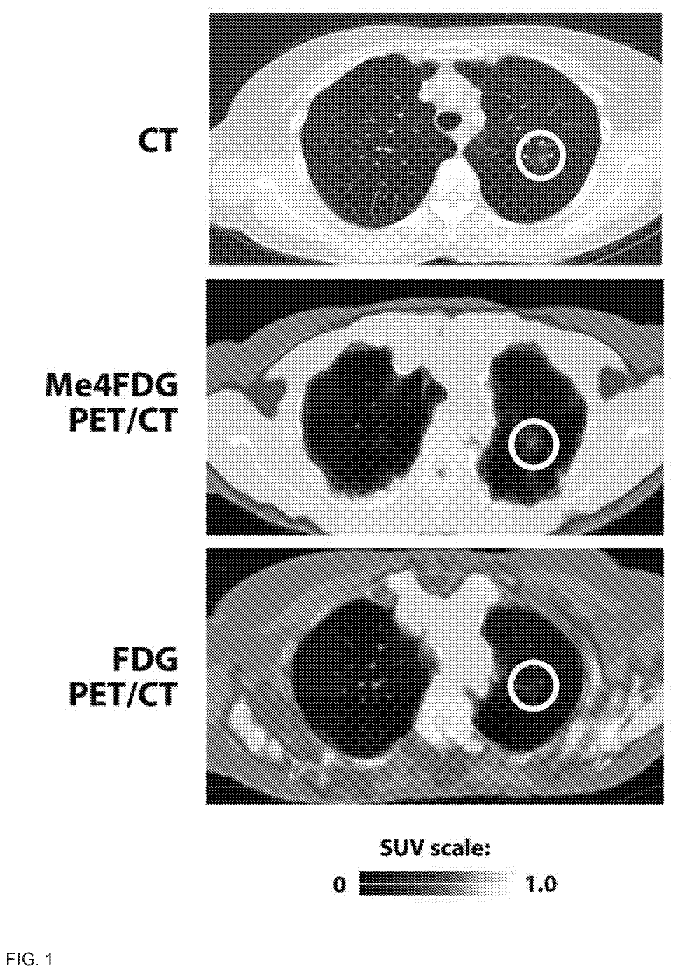

This Example demonstrates the advantages of Me4FDG imaging over conventional FDG, and also shows that the findings described in Example 1 above can be extrapolated successfully to human patients. Six patients were scanned. Four patients had lesions that were positive with Me4FDG. Of these, one had a lesion that was negative with FDG but positive with Me4FDG (FIG. 1).

These results show that the tracer Me4FDG can image some lesions that are negative with the traditional tracer FDG. This type of lesion has the typical appearance of ground-glass opacity, which can correspond to lung adenocarcinoma pre-malignancy. These observations show that lesions that are pre-malignant, yet negative with the traditional tracer FDG, can now be detected. This also shows that the data from the mouse model described in Example 1 is relevant to human patients.

### Example 3

Me4FDG Distinguishes SGLT2-Positive from SGLT2-Negative Lesions

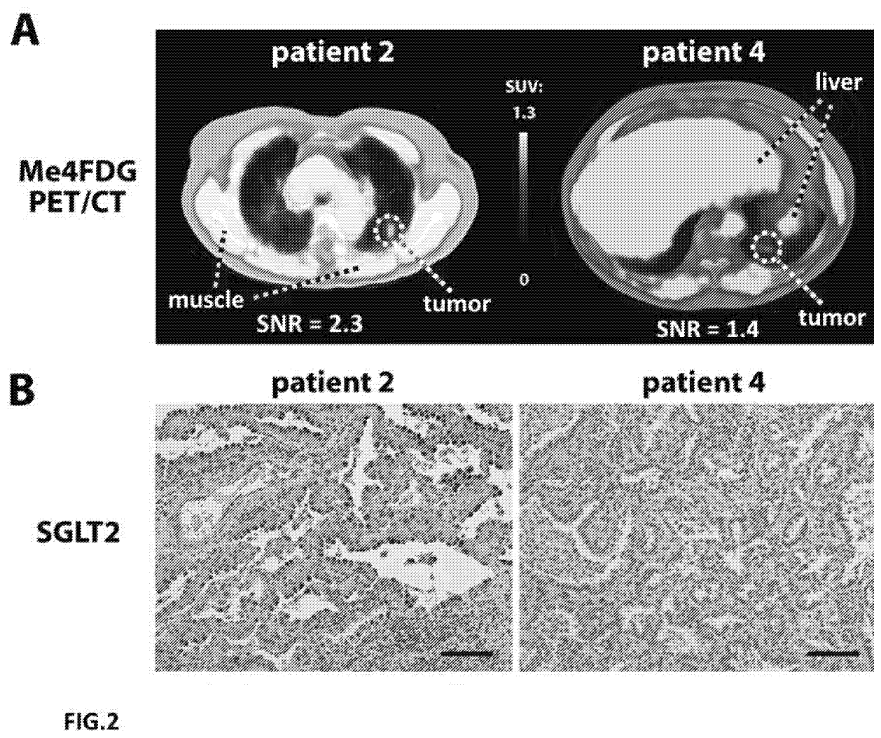

This Example demonstrates a comparison of two lesions in human patients, one positive and one negative with Me4FDG. The patients received a Me4FDG PET/CT and each patient underwent surgery. The pathological specimens were stained with immunohistochemistry to measure the expression of SGLT2 in the lesions. The brown signal indicates the presence of the SGLT2 transporter in the lesion. Both lesions are adenocarcinomas, but while the positive one is positive for SGLT2, the negative one does not have the SGLT2 protein (FIG. 2). This observation confirms that Me4FDG recognizes the lesions that are malignant, and that rely on SGLT2 for glucose uptake.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method of detecting pre-malignant lesions in a subject, the method comprising:
(a) administering to the subject a radiographic tracer for a sodium/glucose cotransporter (SGLT);
(b) performing a radiographic detection scan of the subject; and
(c) detecting signal emitted by the tracer taken up in the scanned subject;
wherein detected signal in the subject is indicative of a pre-malignant lesion.

2. The method of claim 1, wherein the tracer comprises a C 1-O-methyl or ethyl pyranoside having an equatorial hydroxyl group at carbon-2, radiolabeled with ¹⁸F, ¹²³I, or ¹²⁴I or a free hexose having an equatorial hydroxyl group at carbon-2, radiolabeled with ¹⁸F, ¹²³I, or ¹²⁴I.

3. The method of claim 1, wherein the tracer comprises methyl-4-deoxy-4-[¹⁸F]fluoro-D-glucopyranoside (Me4FDG), 1-[¹⁸F]fluoro-1-deoxy-D-glucose (“1-FDG”), or 4-[¹⁸F]fluoro-dapagliflozin.

4. The method of claim 1, wherein the radiographic detection scan is a positron emission tomography (PET) scan.

5. The method of claim 4, wherein the detecting of step (c) comprises calculating a contrast to noise ratio (CNR) of a PET signal.

6. The method of any of claim 1 further comprising administering an inhibitor of sodium-glucose transporter 2 (SGLT2).

7. The method of claim 1, wherein the lesion is a lung, prostate, bladder, breast, or pancreatic lesion.

8. The method of claim 7, wherein the lung lesion comprises atypical adenomatous hyperplasia (AAH), adenocarcinoma in situ, invasive adenocarcinoma, and/or minimally invasive adenocarcinoma.

9. A method of inhibiting the development or progression of a pre-malignant lesion in a subject, the method comprising administering to the subject an inhibitor of SGLT2.

10. A method of treating a pre-malignant lesion in a subject, the method comprising:
(a) administering to the subject a radiographic tracer for a sodium/glucose cotransporter (SGLT);
(b) performing a radiographic detection scan of the subject;
(c) detecting signal emitted by the tracer taken up in the scanned subject; and
(d) administering an inhibitor of sodium-glucose transporter 2 (SGLT2) to the subject.

11. The method of claim 6, wherein the inhibitor is a gliflozin.

12. The method of claim 11, wherein the gliflozin is dapagliflozin, canagliflozin, empagliflozin, or ertugliflozin.

13. The method of claim 10, wherein the tracer comprises a C 1-O-methyl or ethyl pyranoside having an equatorial hydroxyl group at carbon-2, radiolabeled with ¹⁸F, ¹²³I, or ¹²⁴I or a free hexose having an equatorial hydroxyl group at carbon-2, radiolabeled with ¹⁸F, ¹²³I, or ¹²⁴I.

14. The method of claim 10, wherein the tracer comprises methyl-4-deoxy-4-[¹⁸F]fluoro-D-glucopyranoside (Me4FDG), 1-[¹⁸F]fluoro-1-deoxy-D-glucose (“1-FDG”), or 4-[¹⁸F]fluoro-dapagliflozin.

15. The method of claim 10, wherein the radiographic detection scan is a positron emission tomography (PET) scan.

16. The method of claim 15, wherein the detecting of step (c) comprises calculating a contrast to noise ratio (CNR) of a PET signal.

17. The method of claim 10, wherein the lesion is a lung, prostate, bladder, breast, or pancreatic lesion.

18. The method of claim 17, wherein the lung lesion comprises atypical adenomatous hyperplasia (AAH), adenocarcinoma in situ, invasive adenocarcinoma, and/or minimally invasive adenocarcinoma.

19. The method of claim 10, wherein the subject is a human.


SEQUENCE LISTING (1 sequence)

SEQ ID NO: 1 - protein, 47 aa, Homo sapiens
FHEVGGYSGLFDKYLGAATSLTVSEDPAVGNISSFCYRPRPDSYHLL